(19) United States
(12) Patent Application Publication
(10) Pub. No.: US 2024/0406346 A1
(43) Pub. Date: Dec. 5, 2024

(54) COMMUNICATION PLATFORM FOR WALK AND TALK COMMUNICATION SESSIONS

(71) Applicant: Emmanuel Jaikumar John Durai, Newport Beach, CA (US)

(72) Inventor: Emmanuel Jaikumar John Durai, Newport Beach, CA (US)

(21) Appl. No.: 18/731,004

(22) Filed: May 31, 2024

Related U.S. Application Data
(60) Provisional application No. 63/470,136, filed on May 31, 2023.
(60) Provisional application No. 63/528,254, filed on Jul. 21, 2023.
(60) Provisional application No. 63/528,271, filed on Jul. 21, 2023.
(60) Provisional application No. 63/528,275, filed on Jul. 21, 2023.

(51) Int. Cl. H04N 7/14; A61B 5/11; G06Q 10/1093; G16H 80/00

(52) U.S. Cl. CPC H04N 7/147; A61B 5/1118; G06Q 10/1095; H04N 7/142; G16H 80/00

(57) ABSTRACT

A method comprising using at least one hardware processor to: receive an indication for desire for a session with a professional; confirm a professional's availability; schedule session; receive indication to begin session through a platform; activate virtual walking video meeting, by activating video call; receive information at least one of a pedometer and activity tracker; and communicate information to the platform.

### RELATED APPLICATIONS INFORMATION

The application claims priority under 35 U.S.C. § 119 (c) to U.S. Provisional Patent Application No. 63/470,136, entitled “Systems and Methods for Anxiety and Depression Therapy,” filed May 31, 2023, U.S. Provisional Patent Application No. 63/528,254, entitled “Communication Platform for Walk and Talk Communication Sessions,” filed Jul. 21, 2023, U.S. Provisional Patent Application No. 63/528,271, entitled “Communication Platform for Walk and Talk Communication Sessions with Scoring,” filed Jul. 21, 2023, and U.S. Provisional Patent Application No. 63/528,275, entitled “Communication Platform for Walk and Talk Communication Group Chat Sessions,” filed Jul. 21, 2023, each of which is incorporated herein by reference as if set forth in full.

### BACKGROUND

### Field of the Invention

The embodiments described herein are generally directed to a service delivery model that provides anxiety and depression therapy, and more particularly to a platform that allows connectivity for Walk and Talk sessions.

### Description of the Related Art

According to research by Mckinsey and company, 60% psychiatrists don't accept insurance and 1 out of 4 adults with mental illness don't receive mental health due to affordability. According to MHA (mental health America), almost a third (28.2%) of all adults with a mental illness reported that they were not able to receive the treatment they needed. 42% of adults with AMI (Any me Tal illness) reported they were unable to receive necessary care because they could not afford it. 10.8% (over 5.5 million) of adults with a mental illness are uninsured. MHA is nation's leading community-based nonprofit dedicated to addressing the needs of those living with mental illness and promoting the overall mental health of all.

Research suggests walking practices alleviates depression and anxiety. Anxiety and depression are the widely diagnosed mental health issues in the country. Hence addressing the core issues, solves many issues around society's mental health. One-off-sessions are also often sufficient and address several issues around mental health treatment such as affordability. Some forms of therapy are definitely not suitable for one-off sessions, and some issues need more in-depth work than a single session can possibly provide. However, they are not online and not widely accessible by many.

Also, the research studies point out that there are increasing demand for mental health professionals however, not enough professionals are available to address needs of wider population. Hence a technique like SST (Single Session Therapy) or (SSI Single Session Intervention) is widely beneficial to the society, by putting mental health care readily available to many people.

Overall, for majority of the people, single-session therapy is great! People in general love to talk to someone when they have issues or problems. Research studies show that people are not inclined to talk to relatives or family members about their issues mainly due to the fact that they are not trained or don't like to bog them down with their issues and/or stigma attached to the mental health topic. They like to do it with professionals privately.

In studies, SST has also been shown to help reduce anxiety, recurring nightmares, alcohol abuse and self-harm, as well as help manage phobias and panic attacks.

Between 20 percent and 40 percent of psychotherapy clients stop after a single session, says Michael Hoyt, PhD, a researcher and clinical psychologist with Kaiser Permanente in San Rafael, Calif., and author of “Brief Therapy and Managed Care” (Jossey-Bass, 1995). Though therapists may consider these single sessions failures, in his research Hoyt discovered that clients disagreed. “Most people felt that one or two visits was all they needed,” he says. “The session gave them good ideas, and they took those ideas and ran with them.” In less than two hours, you can get a tune-up and oil change, a tax refund, or a massage—and maybe a new outlook on life. That's the idea behind single-session therapy (SST), a method of counseling in which you show up, talk, listen, learn and leave, possibly forever. “In SST, the therapist and the client approach the meeting as though it will likely be the only one,” says Michael F. Hoyt, PhD.

Trust with therapist and modality that works best for the user is always the main reason there is “matching” phase before deciding on a therapist. This is usually for a short/long term treatment within minimum of 6 weeks being the temporal requirements for most of the modalities. SST will change this. But this is not necessarily the best outcome for low cost anxiety and depression therapy and mental health support becomes available for most of the people in the society.

One of the first therapists who practised single-session therapy (SST) was Sigmund Freud. It is reported that the pioneer of long-term psychoanalysis carried out two well-known single-session treatments with Aurelia Öhm-Kronich (“Katharina”) in 1893 (Freud & Breuer, 1895) and with the famous composer, Gustav Mahler in 1910 (Kuchn, 1965). While there are periodic references to single-session treatment in the literature from that time onwards, with well-known therapists like Alfred Adler, Milton Erickson and Albert Ellis pioneering the use of single therapy sessions often conducted in front of professional and lay audiences, it wasn't until 1990 that the field of SST began to cohere. This is when Moshe Talmon published a book which many, consider seminal and which marked the beginning of a growing interest in SST by appointment or by walk-in. 70-80 percent of people who attend only one session, across a range of therapies, report that the single session was adequate given their current circumstance.

But at present even SST therapy is still not widely available an democratized for people of all income levels.

### SUMMARY

Accordingly, systems, methods, and non-transitory computer-readable media are disclosed to a service delivery model that provides anxiety and depression therapy.

According to one aspect, a method comprising using at least one hardware processor to: receive an indication for desire for a session with a professional; confirm a professional's availability; schedule session; receive indication to begin session through a platform; activate virtual walking video meeting, by activating video call; receive information at least one of a pedometer and activity tracker; and communicate information to the platform.

According to another aspect, a method comprising using at least one hardware processor to: receive an indication for desire for a session with a user; confirm a professional's availability; schedule session; receive indication to begin session through a platform; activate virtual walking video meeting, by activating video call; receive information at least one of a pedometer and activity tracker from a user device through the platform.

It should be understood that any of the features in the methods above may be implemented individually or with any subset of the other features in any combination. Thus, to the extent that the appended claims would suggest particular dependencies between features, disclosed embodiments are not limited to these particular dependencies. Rather, any of the features described herein may be combined with any other feature described herein, or implemented without any one or more other features described herein, in any combination of features whatsoever. In addition, any of the methods, described above and elsewhere herein, may be embodied, individually or in any combination, in executable software modules of a processor-based system, such as a server, and/or in executable instructions stored in a non-transitory computer-readable medium.

### BRIEF DESCRIPTION OF THE DRAWINGS

The details of the present invention, both as to its structure and operation, may be gleaned in part by study of the accompanying drawings, in which like reference numerals refer to like parts, and in which:

FIG. 1 illustrates an example infrastructure, in which one or more of the processes described herein, may be implemented, according to an embodiment;

FIG. 2 illustrates an example processing system, by which one or more of the processes described herein, may be executed, according to an embodiment; and

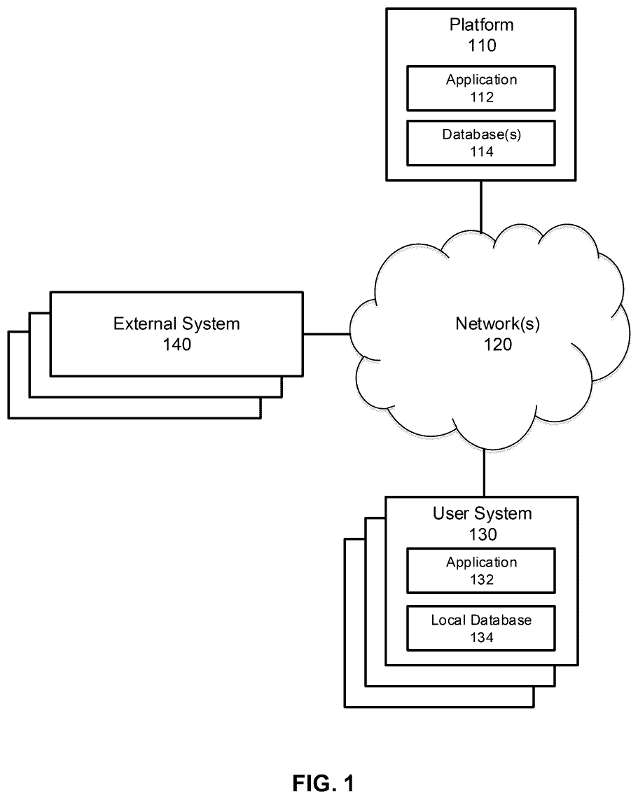

Appendix A includes screen shots for an example user interface that can be implemented in the infrastructure of FIG. 1.

### DETAILED DESCRIPTION

In an embodiment, systems, methods, and non-transitory computer-readable media are disclosed for a service delivery model that provides anxiety and depression therapy.

The systems and methods described herein can provide single session walk and talk therapy via a platform and mobile application to provide access and mental health care to many people instantly in real-time. This can also avoid the stigma attached to “being mentally ill like a patient” by not prolonging the therapy to many weeks or months. SST is simple, with no contracts no minimum weeks no monthly therapy sessions. No red tapes. No appointments and waiting rooms.

Incorporating walking/movement capabilities to a live therapy session without requiring patient and therapist to be in the same place, by providing the tools described herein to therapist and the user, provides tremendous result in anxiety disorders and depression. Research studies are there to back this up where walking alleviates depression and anxiety. The systems and methods described herein provide a therapy session is where a patient can focus on therapy plus walking/monitoring physical movement.

After reading this description, it will become apparent to one skilled in the art how to implement the systems and methods described herein in various alternative embodiments and alternative applications. However, although various embodiments will be described herein, it is understood that these embodiments are presented by way of example and illustration only, and not limitation. As such, this detailed description of various embodiments should not be construed to limit the scope or breadth of the the appended claims.

### 1. System Overview

FIG. 1 illustrates an example infrastructure in which one or more of the disclosed processes may be implemented, according to an embodiment. The infrastructure may comprise a platform 110 (e.g., one or more servers) which hosts and/or executes one or more of the various processes, methods, functions, and/or software modules described herein. Platform 110 may comprise dedicated servers, or may instead be implemented in a computing cloud, in which the resources of one or more servers are dynamically and elastically allocated to multiple tenants based on demand. In either case, the servers may be collocated and/or geographically distributed. Platform 110 may also comprise or be communicatively connected to a server application 112 and/or one or more databases 114. In addition, platform 110 may be communicatively connected to one or more user systems 130 via one or more networks 120. Platform 110 may also be communicatively connected to one or more external systems 140 (e.g., other platforms, websites, etc.) via one or more networks 120.

### 1.1. Infrastructure

Network(s) 120 may comprise the Internet, and platform 110 may communicate with user system(s) 130 through the Internet using standard transmission protocols, such as HyperText Transfer Protocol (HTTP), HTTP Secure (HTTPS), File Transfer Protocol (FTP), FTP Secure (FTPS), Secure Shell FTP (SFTP), and the like, as well as proprietary protocols. While platform 110 is illustrated as being connected to various systems through a single set of network(s) 120, it should be understood that platform 110 may be connected to the various systems via different sets of one or more networks. For example, platform 110 may be connected to a subset of user systems 130 and/or external systems 140 via the Internet, but may be connected to one or more other user systems 130 and/or external systems 140 via an intranet. Furthermore, while only a few user systems 130 and external systems 140, one server application 112, and one set of database(s) 114 are illustrated, it should be understood that the infrastructure may comprise any number of user systems, external systems, server applications, and databases.

User system(s) 130 may comprise any type or types of computing devices capable of wired and/or wireless communication, including without limitation, desktop computers, laptop computers, tablet computers, smart phones or other mobile phones, servers, game consoles, televisions, set-top boxes, electronic kiosks, point-of-sale terminals, and/or the like. Each user system 130 may comprise or be communicatively connected to a client application 132 and/or one or more local databases 134.

Platform 110 may comprise web servers which host one or more websites and/or web services. In embodiments in which a website is provided, the website may comprise a graphical user interface, including, for example, one or more screens (e.g., webpages) generated in HyperText Markup Language (HTML) or other language. Platform 110 transmits or serves one or more screens of the graphical user interface in response to requests from user system(s) 130. In some embodiments, these screens may be served in the form of a wizard, in which case two or more screens may be served in a sequential manner, and one or more of the sequential screens may depend on an interaction of the user or user system 130 with one or more preceding screens. The requests to platform 110 and the responses from platform 110, including the screens of the graphical user interface, may both be communicated through network(s) 120, which may include the Internet, using standard communication protocols (e.g., HTTP, HTTPS, etc.). These screens (e.g., webpages) may comprise a combination of content and elements, such as text, images, videos, animations, references (e.g., hyperlinks), frames, inputs (e.g., textboxes, text areas, checkboxes, radio buttons, drop-down menus, buttons, forms, etc.), scripts (e.g., JavaScript), and the like, including elements comprising or derived from data stored in one or more databases (e.g., database(s) 114) that are locally and/or remotely accessible to platform 110. It should be understood that platform 110 may also respond to other requests from user system(s) 130.

Platform 110 may comprise, be communicatively coupled with, or otherwise have access to one or more database(s) 114. For example, platform 110 may comprise one or more database servers which manage one or more databases 114. Server application 112 executing on platform 110 and/or client application 132 executing on user system 130 may submit data (e.g., user data, form data, etc.) to be stored in database(s) 114, and/or request access to data stored in database(s) 114. Any suitable database may be utilized, including without limitation MySQL™, Oracle™, IBM™, Microsoft SQL™, Access™, PostgreSQL™, MongoDB™, and the like, including cloud-based databases and proprietary databases. Data may be sent to platform 110, for instance, using the well-known POST request supported by HTTP, via FTP, and/or the like. This data, as well as other requests, may be handled, for example, by server-side web technology, such as a servlet or other software module (e.g., comprised in server application 112), executed by platform 110.

In embodiments in which a web service is provided, platform 110 may receive requests from user system(s) 130 and/or external system(s) 140, and provide responses in extensible Markup Language (XML), JavaScript Object Notation (JSON), and/or any other suitable or desired format. In such embodiments, platform 110 may provide an application programming interface (API) which defines the manner in which user system(s) 130 and/or external system(s) 140 may interact with the web service. Thus, user system(s) 130 and/or external system(s) 140 (which may themselves be servers), can define their own user interfaces, and rely on the web service to implement or otherwise provide the backend processes, methods, functionality, storage, and/or the like, described herein. For example, in such an embodiment, a client application 132, executing on one or more user system(s) 130, may interact with a server application 112 executing on platform 110 to execute one or more or a portion of one or more of the various functions, processes, methods, and/or software modules described herein.

Client application 132 may be “thin,” in which case processing is primarily carried out server-side by server application 112 on platform 110. A basic example of a thin client application 132 is a browser application, which simply requests, receives, and renders webpages at user system(s) 130, while server application 112 on platform 110 is responsible for generating the webpages and managing database functions. Alternatively, the client application may be “thick,” in which case processing is primarily carried out client-side by user system(s) 130. It should be understood that client application 132 may perform an amount of processing, relative to server application 112 on platform 110, at any point along this spectrum between “thin” and “thick,” depending on the design goals of the particular implementation. In any case, the software described herein, which may wholly reside on either platform 110 (e.g., in which case server application 112 performs all processing) or user system(s) 130 (e.g., in which case client application 132 performs all processing) or be distributed between platform 110 and user system(s) 130 (e.g., in which case server application 112 and client application 132 both perform processing), can comprise one or more executable software modules comprising instructions that implement one or more of the processes, methods, or functions described herein.

### 1.2. Example Processing Device

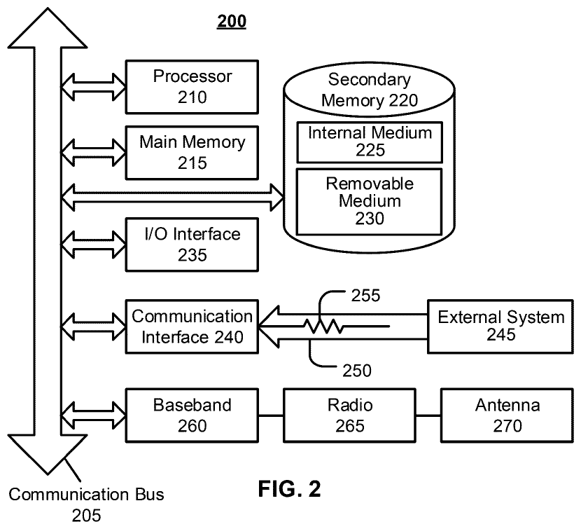

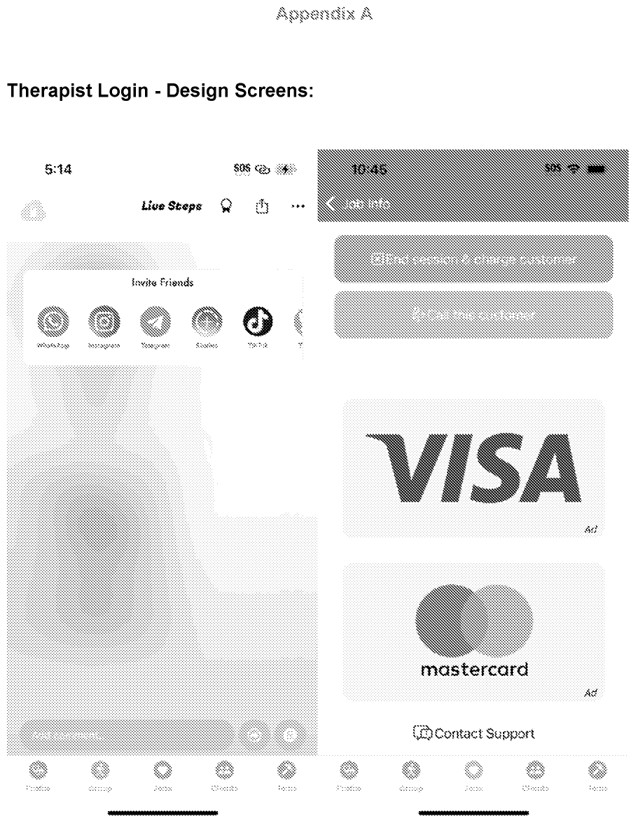

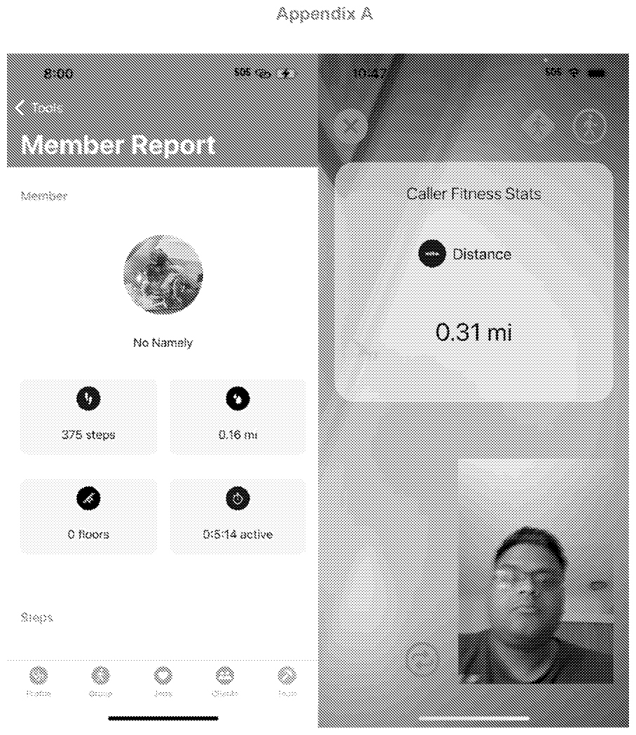

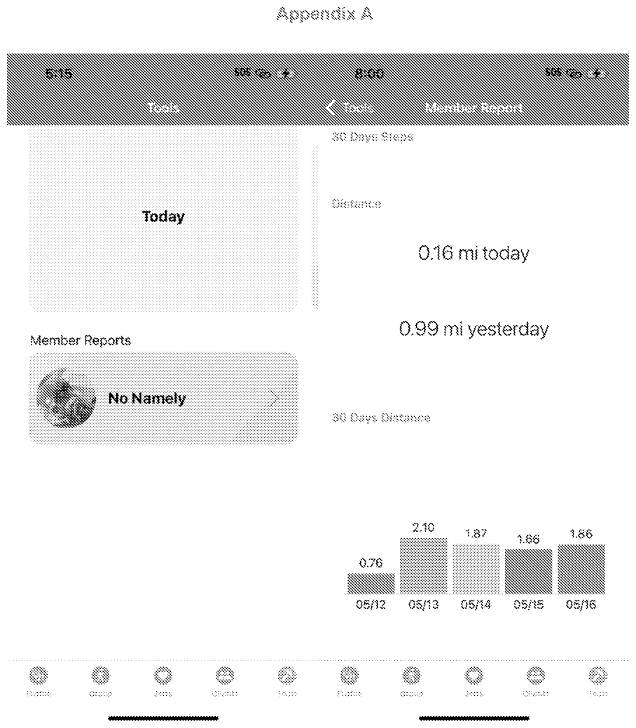

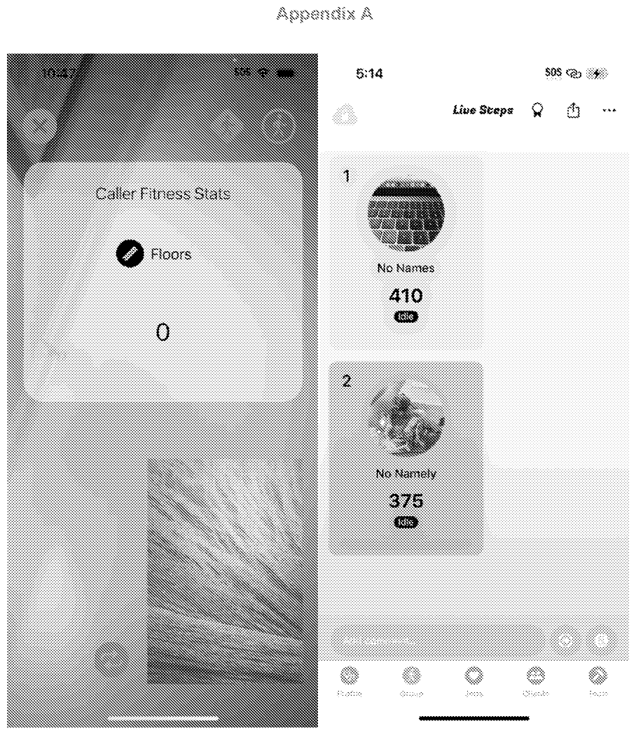

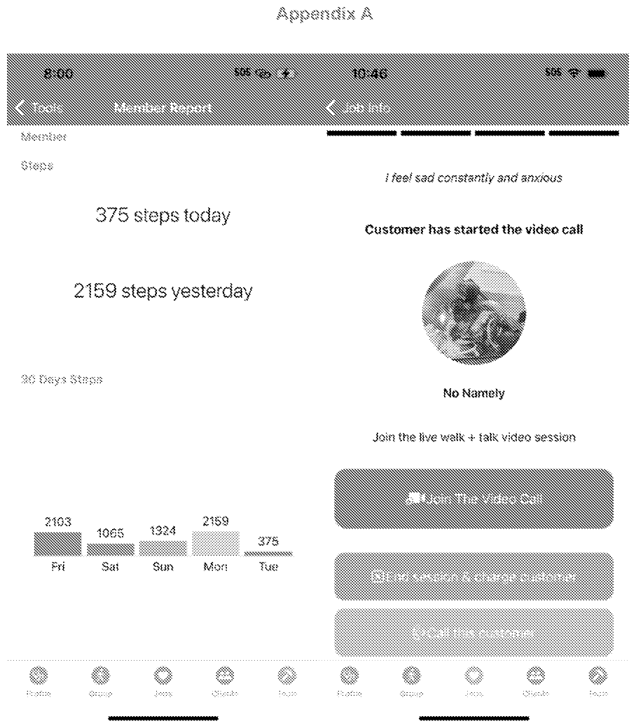

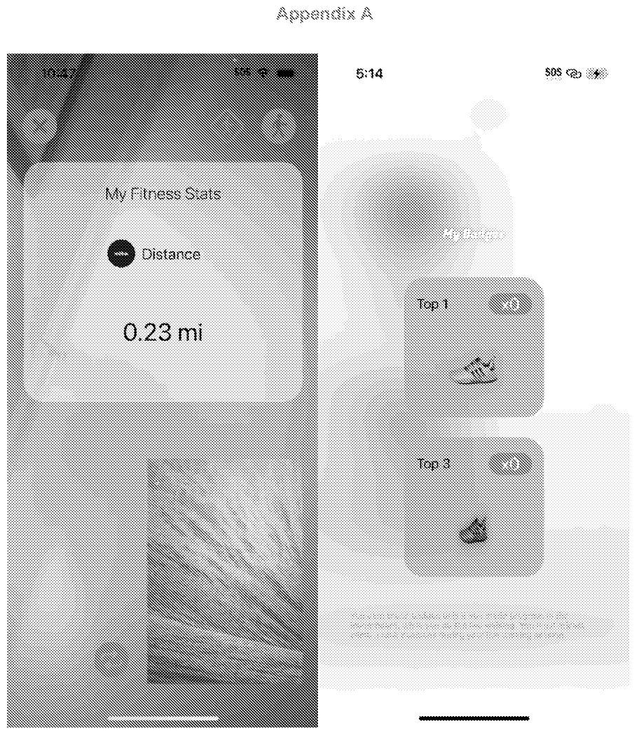

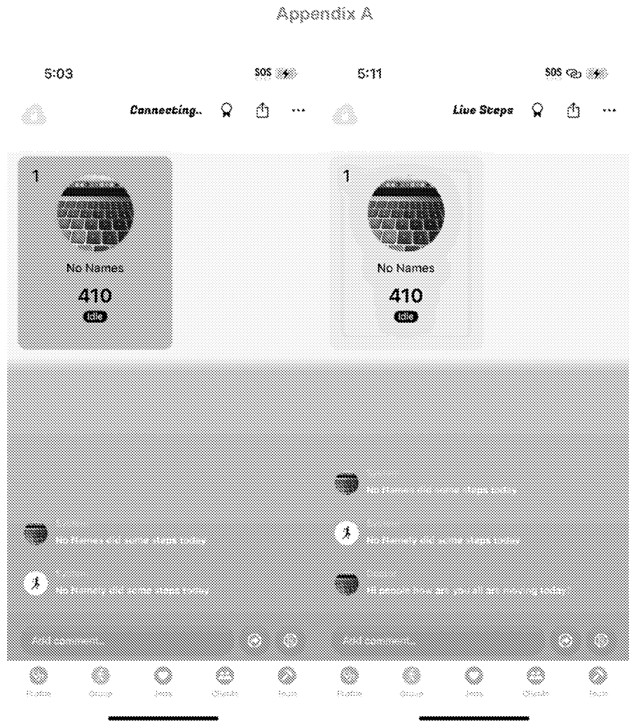

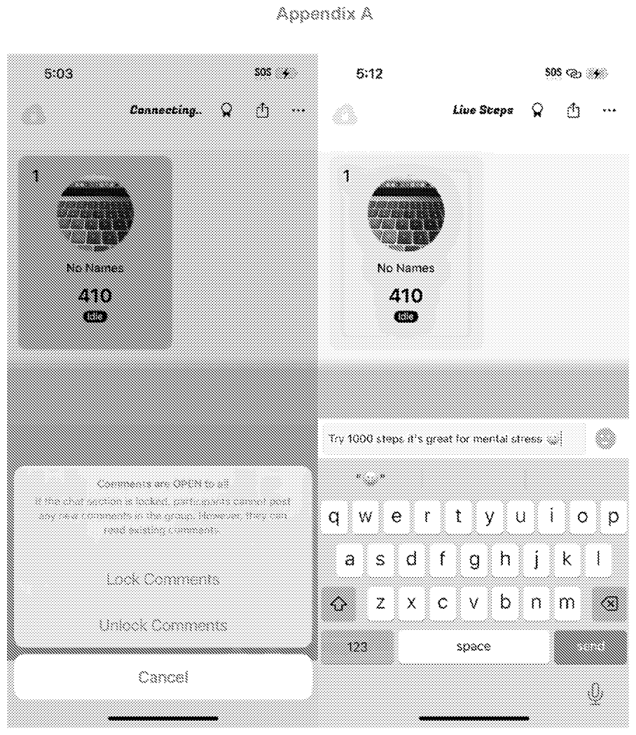

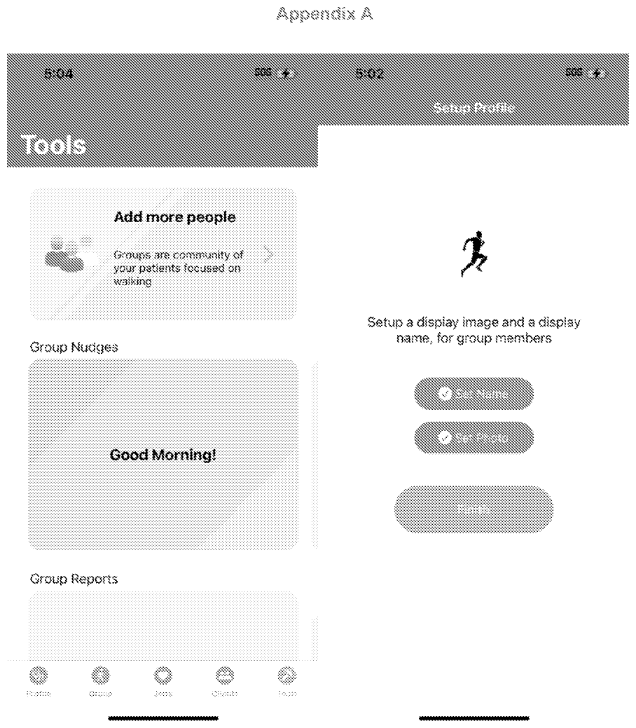

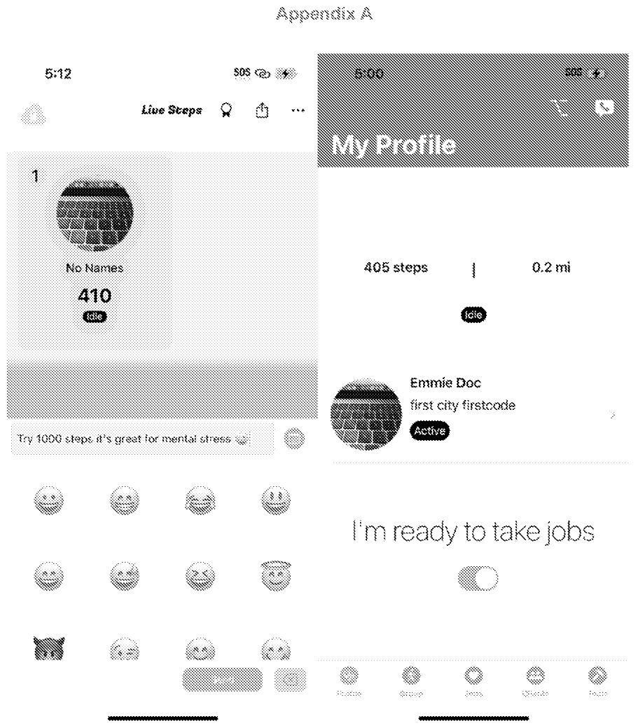

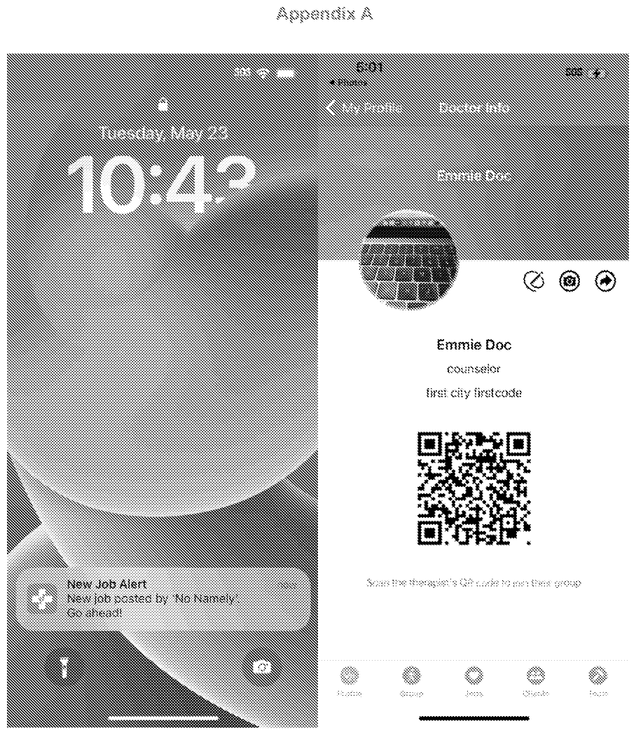

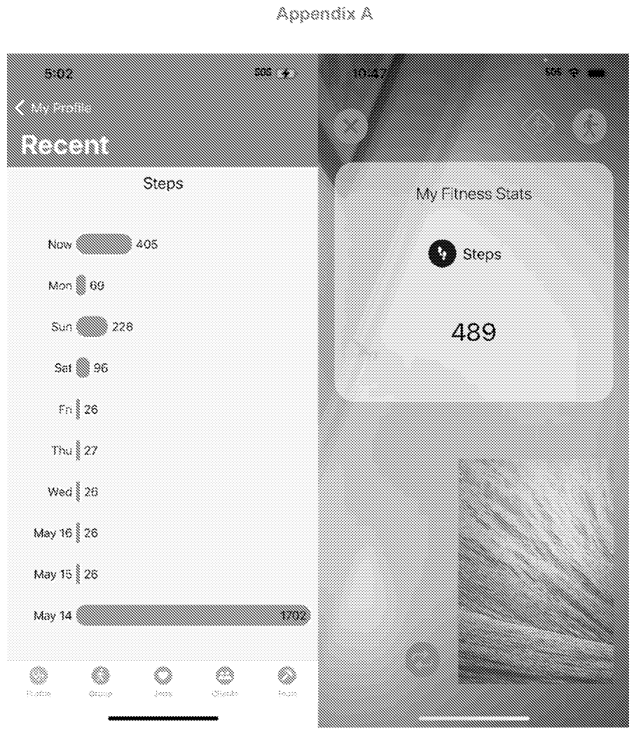

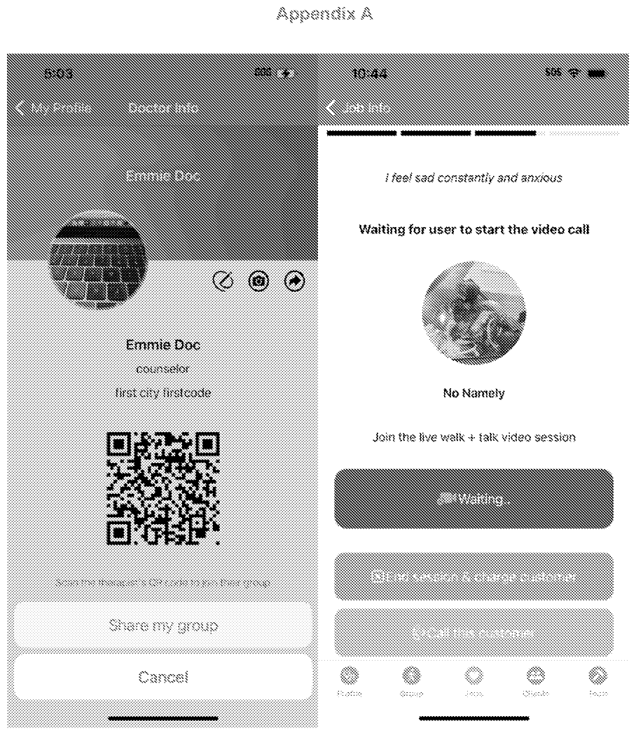

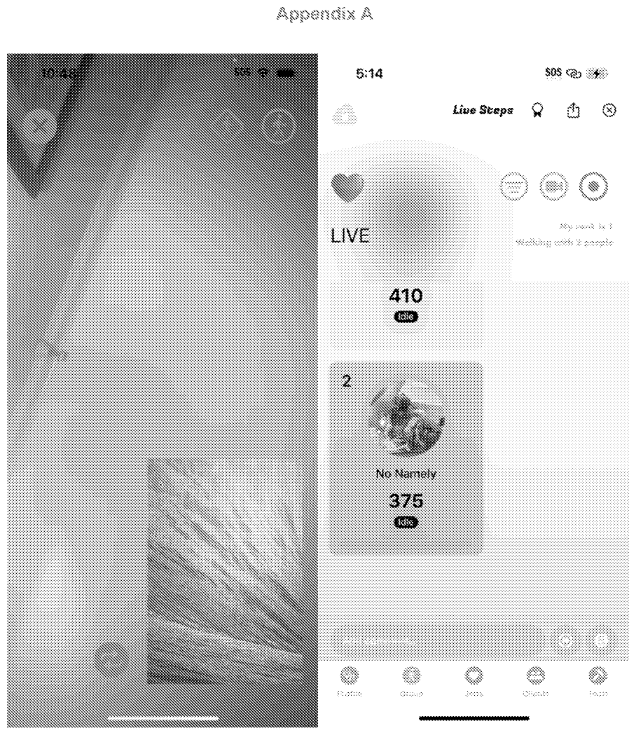

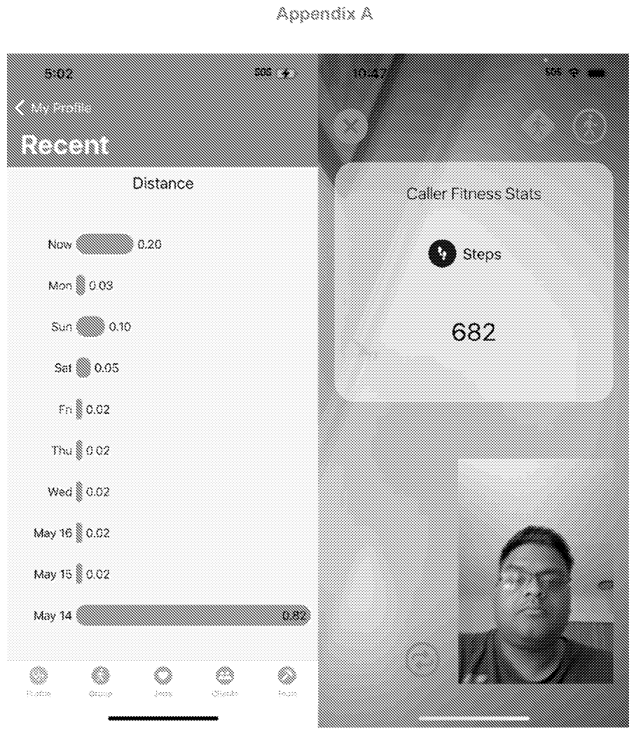

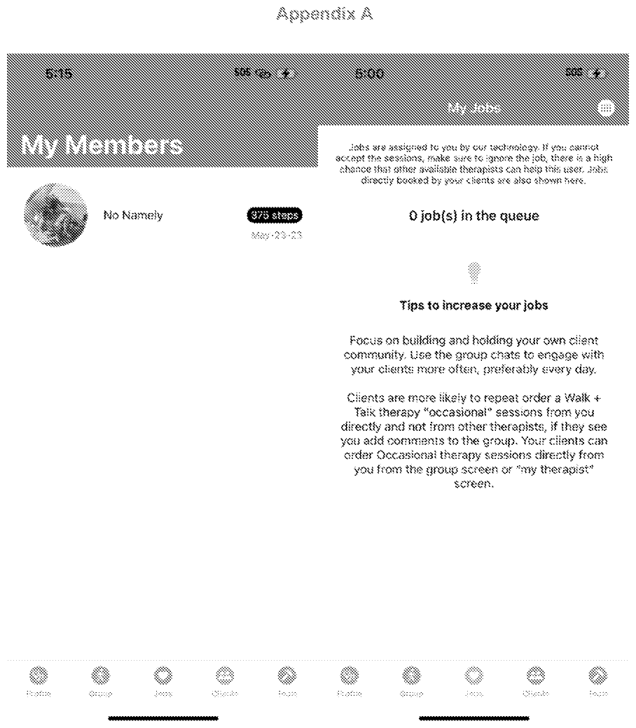

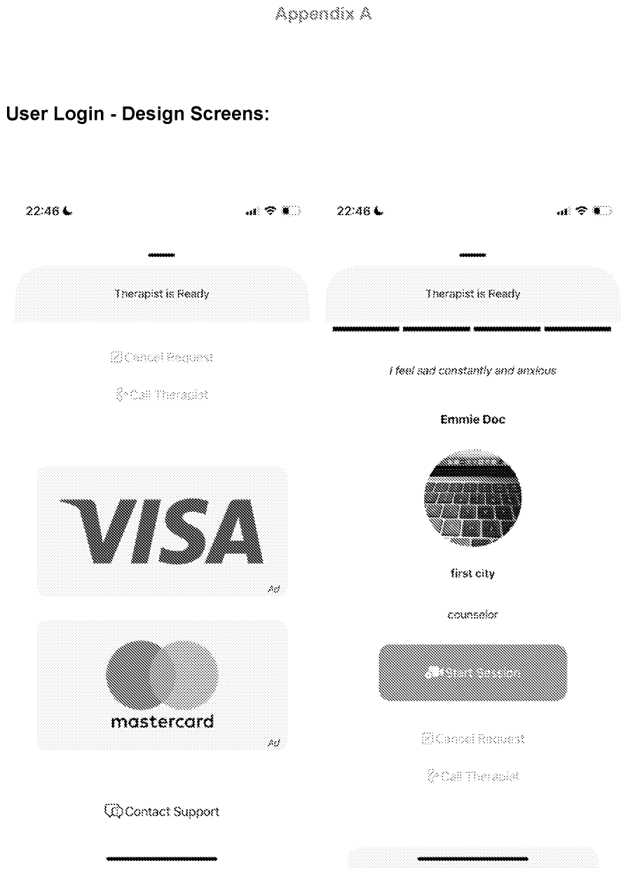

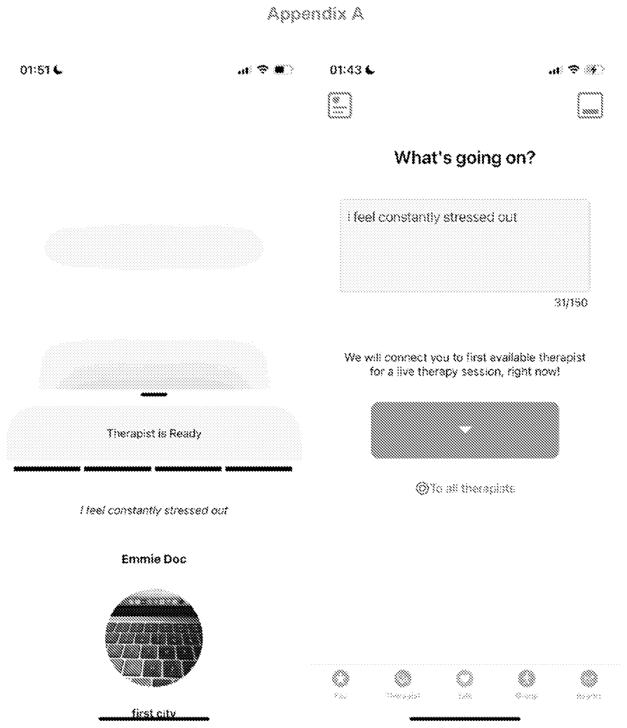

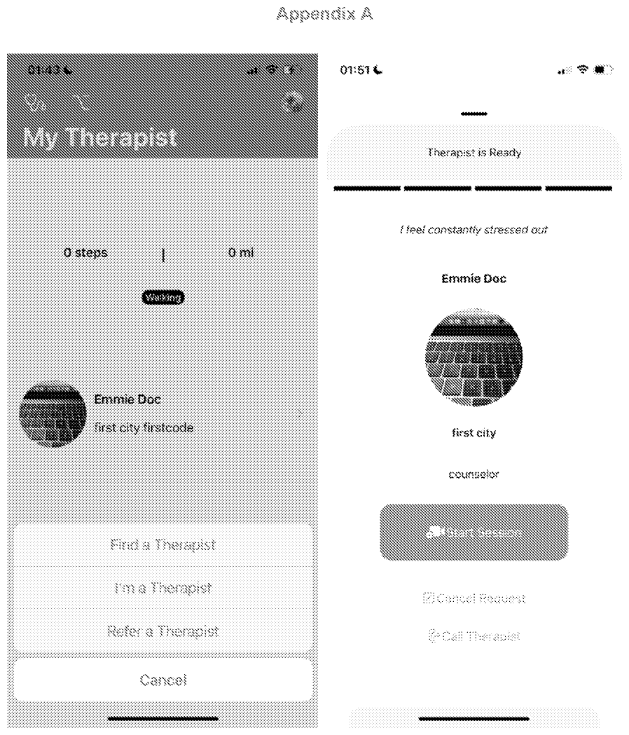

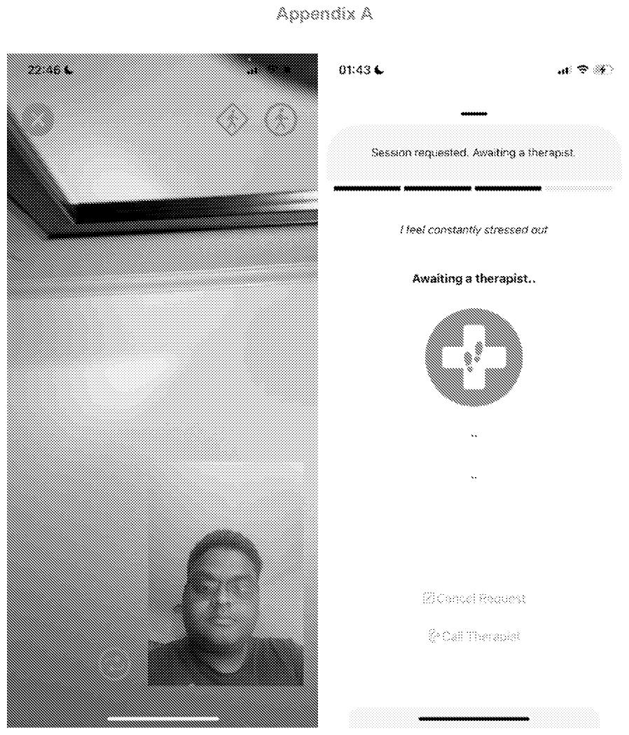

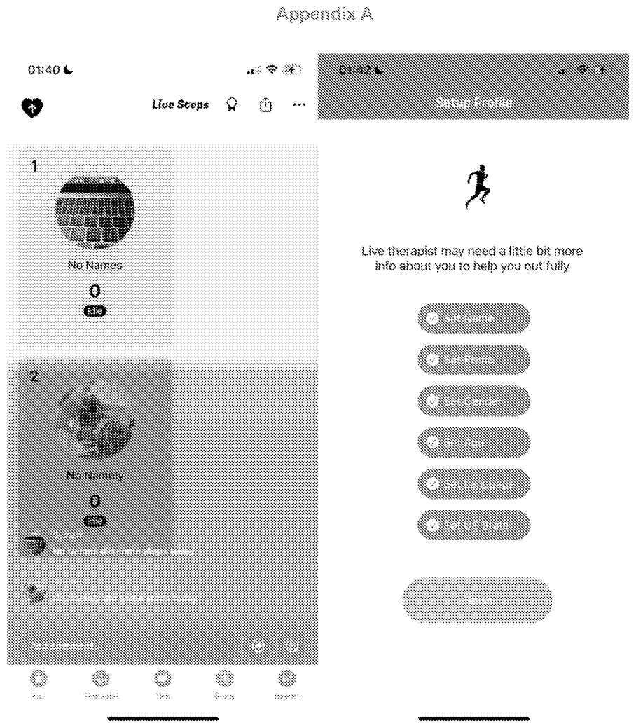

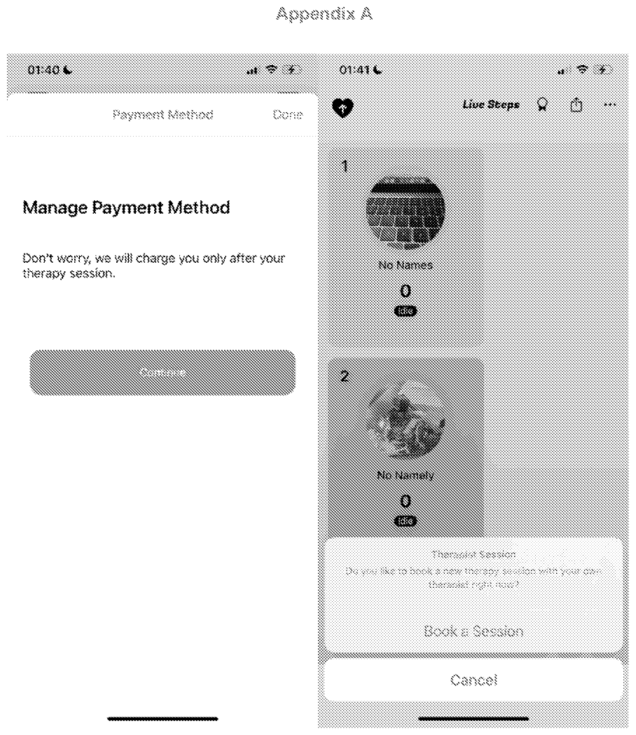

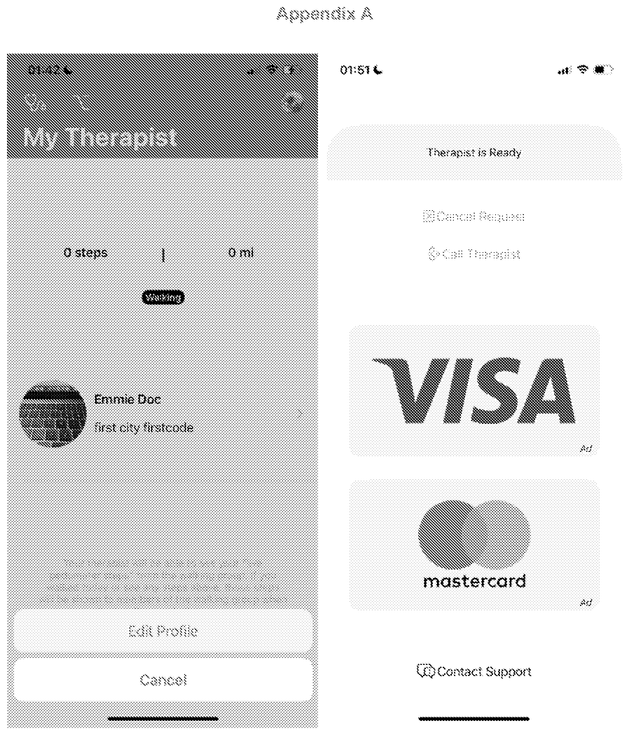

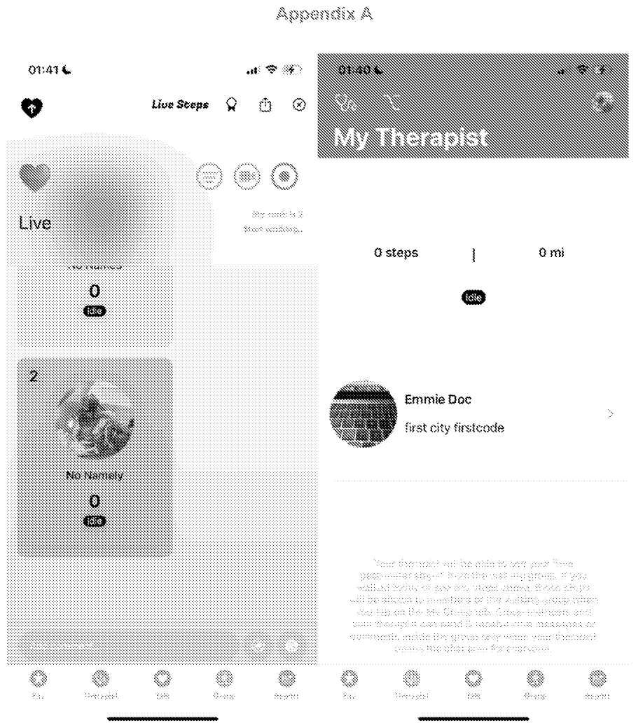

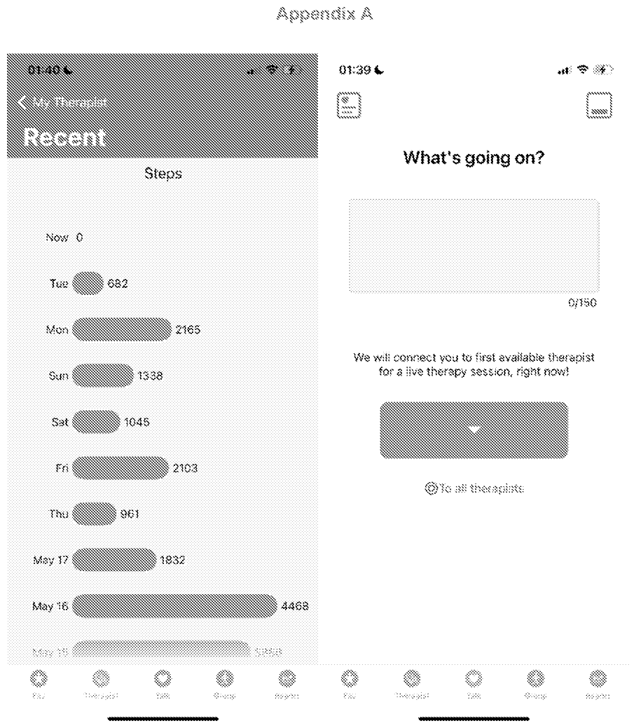

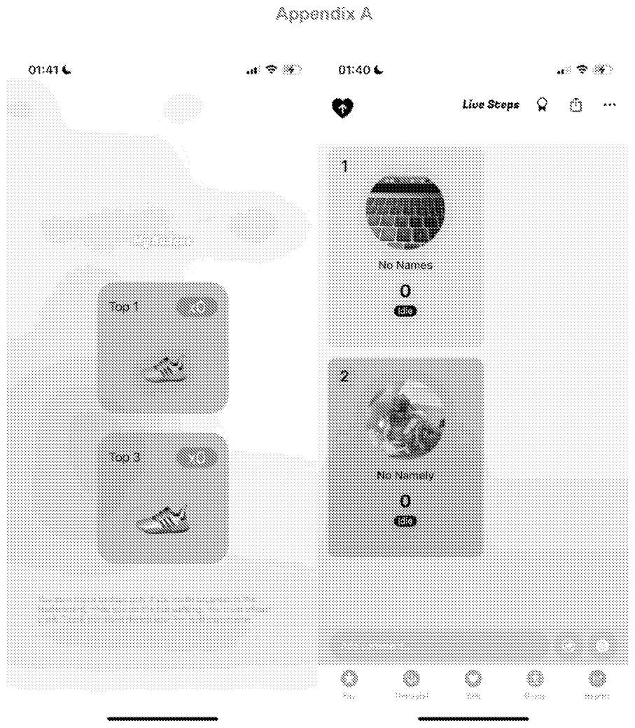

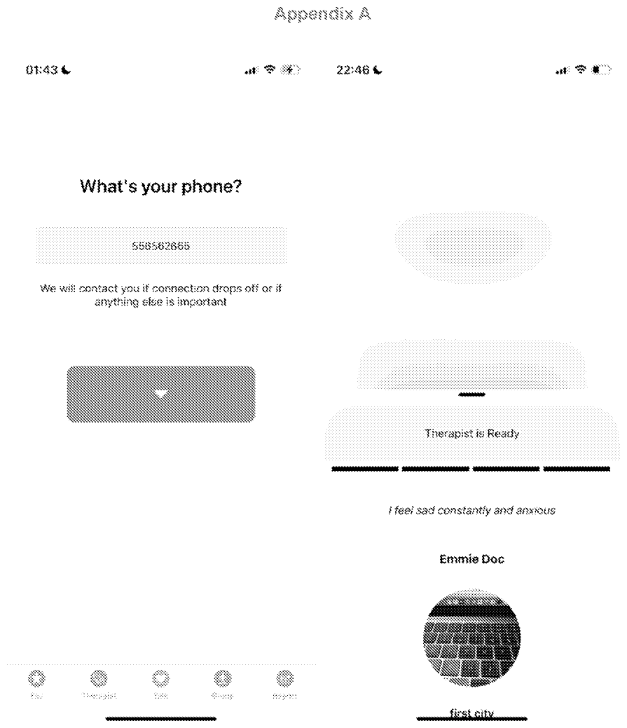

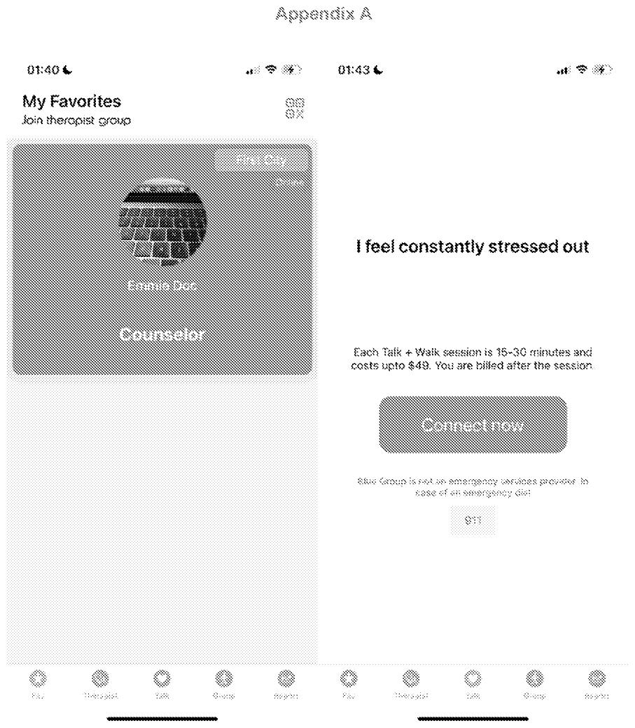

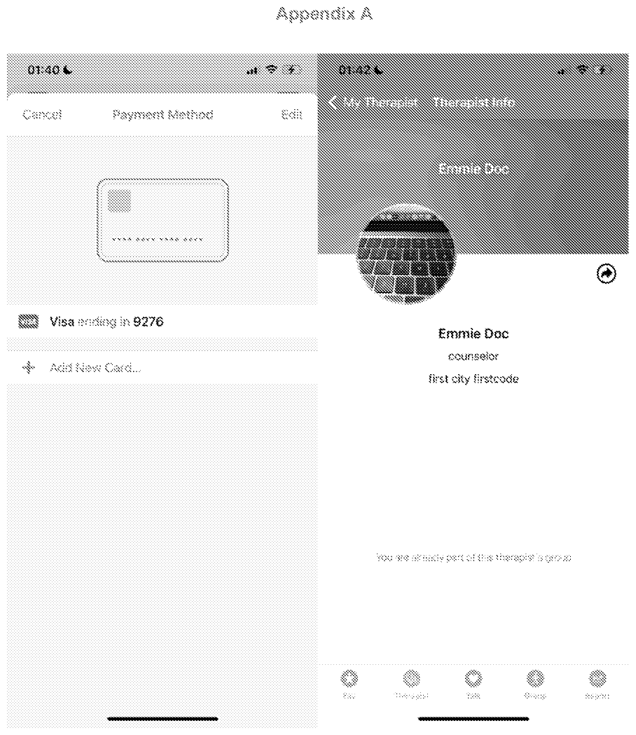

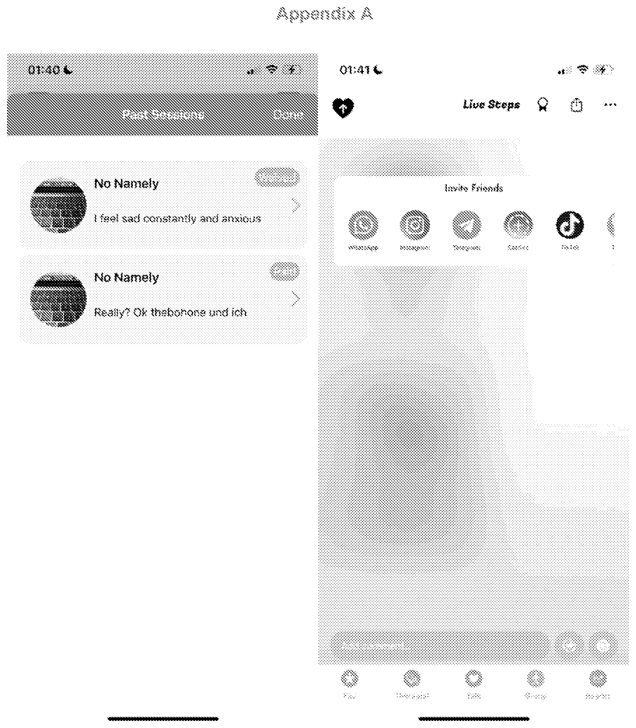

FIG. 2 is a block diagram illustrating an example wired or wireless system 200 that may be used in connection with various embodiments described herein. For example, system 200 may be used as or in conjunction with one or more of the processes, methods, or functions (e.g., to store and/or execute the software) described herein, and may represent components of platform 110, user system(s) 130, external system(s) 140, and/or other processing devices described herein. System 200 can be any processor-enabled device (e.g., server, personal computer, etc.) that is capable of wired or wireless data communication. Other processing systems and/or architectures may also be used, as will be clear to those skilled in the art.

System 200 may comprise one or more processors 210. Processor(s) 210 may comprise a central processing unit (CPU). Additional processors may be provided, such as a graphics processing unit (GPU), an auxiliary processor to manage input/output, an auxiliary processor to perform floating-point mathematical operations, a special-purpose microprocessor having an architecture suitable for fast execution of signal-processing algorithms (e.g., digital-signal processor), a subordinate processor (e.g., back-end processor), an additional microprocessor or controller for dual or multiple processor systems, and/or a coprocessor. Such auxiliary processors may be discrete processors or may be integrated with a main processor 210. Examples of processors which may be used with system 200 include, without limitation, any of the processors (e.g., Pentium™, Core i7™, Core i9™, Xeon™, etc.) available from Intel Corporation of Santa Clara, California, any of the processors available from Advanced Micro Devices, Incorporated (AMD) of Santa Clara, California, any of the processors (e.g., A series, M series, etc.) available from Apple Inc. of Cupertino, any of the processors (e.g., Exynos™) available from Samsung Electronics Co., Ltd., of Seoul, South Korea, any of the processors available from NXP Semiconductors N.V. of Eindhoven, Netherlands, and/or the like.

Processor(s) 210 may be connected to a communication bus 205. Communication bus 205 may include a data channel for facilitating information transfer between storage and other peripheral components of system 200. Furthermore, communication bus 205 may provide a set of signals used for communication with processor 210, including a data bus, address bus, and/or control bus (not shown). Communication bus 205 may comprise any standard or non-standard bus architecture such as, for example, bus architectures compliant with industry standard architecture (ISA), extended industry standard architecture (EISA), Micro Channel Architecture (MCA), peripheral component interconnect (PCI) local bus, standards promulgated by the Institute of Electrical and Electronics Engineers (IEEE) including IEEE 488 general-purpose interface bus (GPIB), IEEE 696/S-100, and/or the like.

System 200 may comprise main memory 215. Main memory 215 provides storage of instructions and data for programs executing on processor 210, such as any of the software discussed herein. It should be understood that programs stored in the memory and executed by processor 210 may be written and/or compiled according to any suitable language, including without limitation C/C++, Java, JavaScript, Perl, Python, Visual Basic, .NET, and the like. Main memory 215 is typically semiconductor-based memory such as dynamic random access memory (DRAM) and/or static random access memory (SRAM). Other semiconductor-based memory types include, for example, synchronous dynamic random access memory (SDRAM), Rambus dynamic random access memory (RDRAM), ferroelectric random access memory (FRAM), and the like, including read only memory (ROM).

System 200 may comprise secondary memory 220. Secondary memory 220 is a non-transitory computer-readable medium having computer-executable code and/or other data (e.g., any of the software disclosed herein) stored thereon. In this description, the term “computer-readable medium” is used to refer to any non-transitory computer-readable storage media used to provide computer-executable code and/or other data to or within system 200. The computer software stored on secondary memory 220 is read into main memory 215 for execution by processor 210. Secondary memory 220 may include, for example, semiconductor-based memory, such as programmable read-only memory (PROM), erasable programmable read-only memory (EPROM), electrically erasable read-only memory (EEPROM), and flash memory (block-oriented memory similar to EEPROM).

Secondary memory 220 may include an internal medium 225 and/or a removable medium 230. Removable medium 230 is read from and/or written to in any well-known manner. Removable storage medium 230 may be, for example, a magnetic tape drive, a compact disc (CD) drive, a digital versatile disc (DVD) drive, other optical drive, a flash memory drive, and/or the like.

System 200 may comprise an input/output (I/O) interface 235. I/O interface 235 provides an interface between one or more components of system 200 and one or more input and/or output devices. Example input devices include, without limitation, sensors, keyboards, touch screens or other touch-sensitive devices, cameras, biometric sensing devices, computer mice, trackballs, pen-based pointing devices, and/or the like. Examples of output devices include, without limitation, other processing systems, cathode ray tubes (CRTs), plasma displays, light-emitting diode (LED) displays, liquid crystal displays (LCDs), printers, vacuum fluorescent displays (VFDs), surface-conduction electron-emitter displays (SEDs), field emission displays (FEDs), and/or the like. In some cases, an input and output device may be combined, such as in the case of a touch panel display (e.g., in a smartphone, tablet computer, or other mobile device).

System 200 may comprise a communication interface 240. Communication interface 240 allows software to be transferred between system 200 and external devices (e.g. printers), networks, or other information sources. For example, computer-executable code and/or data may be transferred to system 200 from a network server (e.g., platform 110) via communication interface 240. Examples of communication interface 240 include a built-in network adapter, network interface card (NIC), Personal Computer Memory Card International Association (PCMCIA) network card, card bus network adapter, wireless network adapter, Universal Serial Bus (USB) network adapter, modem, a wireless data card, a communications port, an infrared interface, an IEEE 1394 fire-wire, and any other device capable of interfacing system 200 with a network (e.g., network(s) 120) or another computing device. Communication interface 240 preferably implements industry-promulgated protocol standards, such as Ethernet IEEE 802 standards, Fiber Channel, digital subscriber line (DSL), asynchronous digital subscriber line (ADSL), frame relay, asynchronous transfer mode (ATM), integrated digital services network (ISDN), personal communications services (PCS), transmission control protocol/Internet protocol (TCP/IP), serial line Internet protocol/point to point protocol (SLIP/PPP), and so on, but may also implement customized or non-standard interface protocols as well.

Software transferred via communication interface 240 is generally in the form of electrical communication signals 255. These signals 255 may be provided to communication interface 240 via a communication channel 250 between communication interface 240 and an external system 245 (e.g., which may correspond to an external system 140, an external computer-readable medium, and/or the like). In an embodiment, communication channel 250 may be a wired or wireless network (e.g., network(s) 120), or any variety of other communication links. Communication channel 250 carries signals 255 and can be implemented using a variety of wired or wireless communication means including wire or cable, fiber optics, conventional phone line, cellular phone link, wireless data communication link, radio frequency (“RF”) link, or infrared link, just to name a few.

Computer-executable code is stored in main memory 215 and/or secondary memory 220. Computer-executable code can also be received from an external system 245 via communication interface 240 and stored in main memory 215 and/or secondary memory 220. Such computer-executable code, when executed, enable system 200 to perform the various functions of the disclosed embodiments as described elsewhere herein.

In an embodiment that is implemented using software, the software may be stored on a computer-readable medium and initially loaded into system 200 by way of removable medium 230, I/O interface 235, or communication interface 240. In such an embodiment, the software is loaded into system 200 in the form of electrical communication signals 255. The software, when executed by processor 210, preferably causes processor 210 to perform one or more of the processes and functions described elsewhere herein.

System 200 may comprise wireless communication components that facilitate wireless communication over a voice network and/or a data network (e.g., in the case of user system 130). The wireless communication components comprise an antenna system 270, a radio system 265, and a baseband system 260. In system 200, radio frequency (RF) signals are transmitted and received over the air by antenna system 270 under the management of radio system 265.

In an embodiment, antenna system 270 may comprise one or more antennae and one or more multiplexors (not shown) that perform a switching function to provide antenna system 270 with transmit and receive signal paths. In the receive path, received RF signals can be coupled from a multiplexor to a low noise amplifier (not shown) that amplifies the received RF signal and sends the amplified signal to radio system 265.

In an alternative embodiment, radio system 265 may comprise one or more radios that are configured to communicate over various frequencies. In an embodiment, radio system 265 may combine a demodulator (not shown) and modulator (not shown) in one integrated circuit (IC). The demodulator and modulator can also be separate components. In the incoming path, the demodulator strips away the RF carrier signal leaving a baseband receive audio signal, which is sent from radio system 265 to baseband system 260.

If the received signal contains audio information, then baseband system 260 decodes the signal and converts it to an analog signal. Then the signal is amplified and sent to a speaker. Baseband system 260 also receives analog audio signals from a microphone. These analog audio signals are converted to digital signals and encoded by baseband system 260. Baseband system 260 also encodes the digital signals for transmission and generates a baseband transmit audio signal that is routed to the modulator portion of radio system 265. The modulator mixes the baseband transmit audio signal with an RF carrier signal, generating an RF transmit signal that is routed to antenna system 270 and may pass through a power amplifier (not shown). The power amplifier amplifies the RF transmit signal and routes it to antenna system 270, where the signal is switched to the antenna port for transmission.

Baseband system 260 is communicatively coupled with processor(s) 210, which have access to memory 215 and 220. Thus, software can be received from baseband processor 260 and stored in main memory 210 or in secondary memory 220, or executed upon receipt. Such software, when executed, can enable system 200 to perform the various functions of the disclosed embodiments.

### 2. Process Overview

Embodiments of processes for a service delivery model that provides anxiety and depression therapy. will now be described in detail. It should be understood that the described processes may be embodied in one or more software modules that are executed by one or more hardware processors (e.g., processor 210), for example, as a software application (e.g., server application 112, client application 132, and/or a distributed application comprising both server application 112 and client application 132), which may be executed wholly by processor(s) of platform 110, wholly by processor(s) of user system(s) 130, or may be distributed across platform 110 and user system(s) 130, such that some portions or modules of the software application are executed by platform 110 and other portions or modules of the software application are executed by user system(s) 130. The described processes may be implemented as instructions represented in source code, object code, and/or machine code. These instructions may be executed directly by hardware processor(s) 210, or alternatively, may be executed by a virtual machine operating between the object code and hardware processor(s) 210. In addition, the disclosed software may be built upon or interfaced with one or more existing systems.

Alternatively, the described processes may be implemented as a hardware component (e.g., general-purpose processor, integrated circuit (IC), application-specific integrated circuit (ASIC), digital signal processor (DSP), field-programmable gate array (FPGA) or other programmable logic device, discrete gate or transistor logic, etc.), combination of hardware components, or combination of hardware and software components. To clearly illustrate the interchangeability of hardware and software, various illustrative components, blocks, modules, circuits, and steps are described herein generally in terms of their functionality. Whether such functionality is implemented as hardware or software depends upon the particular application and design constraints imposed on the overall system. Skilled persons can implement the described functionality in varying ways for each particular application, but such implementation decisions should not be interpreted as causing a departure from the scope of the invention. In addition, the grouping of functions within a component, block, module, circuit, or step is for case of description. Specific functions or steps can be moved from one component, block, module, circuit, or step to another without departing from the invention.

Furthermore, while the processes, described herein, are illustrated with a certain arrangement and ordering of subprocesses, each process may be implemented with fewer, more, or different subprocesses and a different arrangement and/or ordering of subprocesses. In addition, it should be understood that any subprocess, which does not depend on the completion of another subprocess, may be executed before, after, or in parallel with that other independent subprocess, even if the subprocesses are described or illustrated in a particular order.

### 2.1. Service Delivery Model

Online therapy has many modalities depending on the requirement of patients. The systems and methods described herein follow a unique and novel approach using a Single Session Therapy (SST) combined with a Walk and Talk therapy to add physical movement to the therapeutic setting. It's all done via, e.g., a platform 110 and user system application 132. SST means users need only one session for their therapy, additional sessions are called “occasional sessions” which are usually not needed. Occasional experience from other modalities may also be used in the therapy sessions, which can include but not limited to, solution focused therapy, CBT (Cognitive behavioral therapy), Integrated CBT, mindfulness based cognitive therapy, DBT (Dialectical behavioral therapy), psycho dynamic therapy, humanistic therapy, holistic therapy and behavioral therapy.

The therapeutic session can be performed while the patient is walking indoors or outdoors. In fact, ideally, all sessions are virtual walking video meetings with stats integrated from a pedometer and activity tracker that can be included in the patient user system 130. Adding physical activity to talk therapy has scientifically proven advantages on depression, anxiety, stress and various other mental health issues. Research points that there is high demand for a walk and talk therapy, but it is hard for the therapists to reach their clients all around the world.

Physical activity tracking like walking and moving is an added benefit to talk therapy. It improves mood in general and adds benefit in addressing depression, anxiety, stress, insomnia and many more mental health issues. Hence, in many implementations all therapy offered with platform 110 are walk and talk therapy. A traditional walk and talk therapy, done physically involves “nature” by walking outdoors with your therapist. However, platform 110 addresses the issue of complexity in physical therapies where commuting and logistics becomes an issue for both therapist and users. Also, the cost constraints in getting people together and therapists rates are not affordable. Hence platform 110 provides an automated, virtual solution that takes things online and it enables users to walk along virtually with a therapist. Therapy sessions are more promising within a therapeutic setting that has a physical activity included. It's a win-win for both therapists and users and to achieve the needed results on the patients.

There are numerous scientific literature explaining the benefits of physical activity in therapy sessions. Walking, running and exercise has been proved very effective to mental health therapy. The therapy session has “talk” component, “physical activity” component, “convenience” component as it happens online, which add value to a conventional/traditional therapy sessions. Platform 110 also addresses the “nature” component by bringing the virtual sessions of walking together into physical world, by enabling user and the therapist to walk along a walking friendly route within their locality if they are closer to each other. If they are connected online, the therapist and users may not be next to each other in the same location, however, they are connected via, e.g., a video call and walking routes via GPS helps users and therapists to follow a path on their own walking routes shown on the app. Both therapist and users would walk along their own safest walking routes outdoors and complete their therapy sessions. Users can store and favorite their walking routes and share it with their therapist or other users in, e.g., group therapy chats. The, online walk and talk solution provides a cost effective and affordable SST experience.

Platform 110 can also integrate the latest technology innovations in smart phones that integrates the activity tracker, video and audio streaming, wearables data, all in one holistic view on therapy sessions, for therapists to make those informed decisions, actions and offer those pertinent and needed care for mental health issues.

In platform 110, therapists are more like independent workers and freelancers or providers of healthcare, who can sign up for an account on platform 110 and simply activate a “switch” indicating they are available to work and ready for sessions. Jobs/sessions posted by users will be reaching available therapists for real-time live walk and talk session. The therapists should be trained and licensed to practice in their state and the state you live in. Licenses and credential verification for all professionals who are capable of providing a psychotherapy like therapists, counselors, doctors and social workers are done appropriately through platform 110. Platform can use third party services, e.g., an external system 140 to integrate and provide such services.

As noted, the therapy modality offered through platform 110 can be called Single session therapy or SST in short. SST is practiced by a lot of therapists in the market as an Integrated CBT or brief therapy or solution focused therapy. Some therapists may also have training in other Talk therapies, those can include CBT and DBT, psycho dynamic therapy, humanistic therapy, holistic therapy and behavioral therapy. They are also an advocate of physical activity working adjunct to mental health improvements. Adding physical activity to traditional therapy is a win win for both client and therapist. Therapy sessions offered through platform 110 are on the go with video on demand via mobile phones, similar to walking video meetings. However, the technology platform can also allow therapists to track user's live steps, distance, active hours, floors, calories and other vitals like weight, height, blood pressure, heart rate, pulse, walking pace, wheel chair motions, etc., e.g., via a phone's pedometer, or other tracker, and Apple Health or other application.

Therapists can also view and monitor the past week or month's performance physical of every client from their own user system application 132

So, how does it typically work? In every real-time live session, that goes about 30 to 60 minutes, the therapist focuses on the patient's strengths and skills, then helps identify and practice things that can help the patient. The therapist also follows a patient's physical activity levels now and in the immediate past and advise on physical activity thresholds if relevant and appropriate. SST also uses principles similar to cognitive-behavioral and solution-focused therapies, both of which are intended to help you fix patient's fix problems or issues. It's more like the therapist looking at what negative thoughts and behaviors might be causing issues, and the therapist look at what positive ones could solve it.

Therapists on platform 110 can, depending on the implementation address the mental health issues as per the age cohorts, e.g., ages 18-25, ages 26-49, ages 50+.

Typically, in platform 110, the patient is connected to the first available walk & talk therapists for only one session, as if they are not going to need anything further. However, SST is not one size fits all approach. There is always provisions to book further sessions with the same or different therapist. But the first session will always be done as if the patient is not going to show up again. Research has shown that 50% of users taking first session don't show up in traditional therapy sessions. 88% users say they got what they want in first 3 months of receiving the one SST session that changed their quality of life and thinking and feelings. They were able to work with what they got in 1 SST session. Thus, as noted platform 110 can also support further follow up sessions as needed. These further sessions are called “Occasional sessions”. If the patient likes the therapists and patient or therapist feels like more sessions are needed, then the patient can decide to join a group associated with the therapist.

The patient's user system application 132 can be configured to show a list of therapists to the patient has used in the past, so they can choose a therapist and join their group. Groups can comprise live walking leaderboards and chat rooms where other clients of the therapists are also present. Therapist and group members can provide comments like chats if appropriate. Chats are moderated/blocked and controlled by therapists, for example: comments allowed only 1 hour a day or only therapists can post comments and not people. The patient can then also book further 1-1 single sessions with your therapist and schedule it for later date once you have joined the therapist group. The groups support live walking leaderboards were all the other participants can all compete with each other or walk along virtually, in real-time.

Research suggests such walking practices alleviates depression and anxiety. Anxiety and depression are the widely diagnosed mental health issues in the country. Hence addressing the core issues, solves many issues around society's mental health.

Some forms of therapy are definitely not suitable for one-off sessions, and some issues need more in-depth work than a single session can possibly provide. The therapist will be able to advise patients about this and suggest a best course of action. However, they are not online, not widely accessible by many. Also the research studies point out that there are increasing demand for mental health professionals however, not professionals are available to address needs of wider population. Hence a technique or service delivery model like SST (Single Session Therapy) or (SSI Single Session Intervention) is widely beneficial to the society, by putting mental health care readily available to many people. Rather than waiting until the time is perfectly right, it makes sense to get help when people need it the most. Blue Group's mission is to be the leader in on-demand therapy for mental health issues starting with depression and anxiety. SST is also known as Integrated CBT by some mental health practitioners.

Research suggests that mental health professionals are in shortage. There is not many out there for everyone and cannot do repeat sessions for everyone. We will address that market gaps with this SST technique. Identifying therapists that closely aligning with and treating patients of anxiety and depression in a shortest possible time is what we want I think. Some training contents available for SST that we can ask our therapists to go through as part of their on-boarding process.

As we incorporate Walking/movement capabilities to the live therapy session by providing tools to therapist and the user, it provides tremendous result in anxiety disorders and depression. Research studies are there to back this up where walking alleviates depression and anxiety. Research also says that although ppl know this, they never do it. A therapy session is were they can focus on therapy plus walking/monitoring physical movement. We bring in all together.

Overall, for majority of the people, single-session therapy is great! People in general love to talk to someone when they have issues or problems. Research studies show that people are not inclined to talk to relatives or family members about their issues mainly sue to the fact that they are not trained or don't like to bog them down with their issues and/or stigma attached to the mental health topic. They like to do it with professionals privately. Blue group single session walk and talk therapy via a simple mobile app, provides access and mental health care to many people instantly in real-time. This can also avoid the stigma attached to “being mentally ill like a patient” by not prolonging the therapy to many weeks or months. SST is simple, with no contracts no minimum weeks no monthly therapy sessions. No red tapes. No appointments and waiting rooms!

In studies, SST has also been shown to help reduce anxiety, recurring nightmares, alcohol abuse and self-harm, as well as help manage phobias and panic attacks.

### 3. User Interface

The user interface of user system application 132, example screens of which are include in the attached Appendix A can be configured to provide an UBER like real-time booking simplicity for booking a therapy session, right now. This allows users/patient to get help when need the most and rather waiting to schedule an appointment and long wait times and high costs. Thus, as illustrated in the example screen shots, the user can log on, select a therapist/session and the patient user system 130, through platform 110 can arrange the session with and available therapist. The user interface can allow the patient to, e.g., simply enter/post what they feel right now or what's the problem right now and get connected to a therapist in real-time and live. Therapy offered is always a single-session therapy with no obligations to continue sessions further. Users are not tied to contracts or weeks of therapy.

In certain embodiments, user system application 132 and/or platform 110 can be configured to use Emotion Artificial Intelligence (AI) to scan the Natural Language Processing (NLP) from what a patient enters/posts to detect emergency signs, warning signs, modality, seriousness, specialities like anxiety, depression etc. AI can also be used to scan the video or audio in real-time and user languages to detect and act upon various user emotions. AI can also be used to match users to the right therapists in real-time based on factors like modalities, therapists location, availability and user's age. AI can also give an automated feedback to therapists. Real-time AI processing can provide hooks to law enforcement and emergency services if needed, for example, a suicidal thought and a post about it immediately triggers an alert to emergency services and to the therapist. AI is not a replacement to human emotions and human touch, but rather complements the therapeutic process. In psychological terms, everything that helps to create a therapeutic alliance, such as emotional intelligence and social intelligence, is not going to be replaced by any AI in the architecture of FIG. 1. Hence a real person/therapist will always listen to the full problem posted by the users. Other AI and other advanced algorithms can be used in job scheduling to allocated jobs to right available therapists. See below for more details.

The Emotion AI can be configured to give a score to every user like a risk score or profile score of symptoms of anxiety or depression. The score can be a helpful diagnosis for the therapists to act upon during the session. The AI score and analysis will be seen by the therapist in the job screen (see Appendix) while picking up job requests that are posted on the platform 110. While the AI Models are trained for anxiety and depression symptoms, the AI score does not need 100% accuracy. Emotion AI reads user text, audio, video and analyses human emotions, feeling, sadness, depression, mood etc. And provide a score about every user who requests a real-time live therapy to a therapist, before they even start a therapy session.

In certain embodiments, scores range from 1 to 10. It's an indicative figure for therapist to know how likely the patient/user is experiencing depression or anxiety. A deep learning algorithm can be deployed to train the AI models to include training for depression, anxiety, feelings, human emotions like happy, angry, sadness etc. Platform 110 can be trained to read text, live audio and live video to analyze patterns of depression and anxiety and other mental health issues and human emotions.

With regards to SST, the technology platform support overlaying details on top of the user video or audio calls. Every session can be timed and there can also be a timer reminder to close portion of sessions like beginning, intermediate and ending. This is to enable SST process easy for therapists to keep an eye on a reminder or a set timer on the screen. Technology for fitness tracking like steps, distance, heart rate, etc. can be overlaid on the session screen along with SST parameters. Session screen can be audio only, text only or audio vide only or all 3 audio, video and text combined together.

### 4. Platform

Thus, platform 110 combined with use system applications 132 can provide on demand therapy for anxiety and depression, where users can, depending on the implementation, pay one flat fee, with no intake forms, no red tape, no contracts. The services provided by platform 110 provide on demand, as and when it's needed, online therapy with primarily single session therapy (SST) or single session intervention (SSI). By keeping the therapy short or even possible to one single session. At the core of this SST/SSI is talk therapy with optional physical movement like walking is added to the therapeutic session. To keep patients focused on movement, they can join a simple walking group, where therapist can monitor their movement statistics.

In that regard, therapy is typically an individual therapy and not group therapy although a group step challenge can be provided. In certain implementations, each therapist has their own group/clients participating in the step challenge (sense of community to keep walking/moving going on). This group can be extended into group therapy in certain implementations. Platform 110 can also be configured to provide one-on-one chats with a primary therapist, where, e.g., therapist responds at-least once a day.

Platform 110 can integrate data from an activity tracker, video and audio streaming, wearables data, in one holistic view on online real-time therapy sessions, that can be used by therapists to make more informed decisions, actions and offer the pertinent the needed care for (physical and—needed for patent) mental health issues.

Platform 110 can also provide job scheduling considering various factors on therapists availability, location, current demand, pricing, customer mood, customer age, sex, language, therapists filtering based on even distribution to provide equal opportunity to all therapists or to have best outcome for the customers in job distribution. For example, an experienced therapist connected to customer based on the estimated complexity of the issue presented in the job screen by the customer.

As noted, platform 110 also comprises AI (Artificial Intelligence) with deep learning. For example, Emotion AI, which refers to artificial intelligence that detects and interprets human emotional signals in text (using natural language processing and sentiment analysis), audio (using voice emotion AI), video (using facial movement analysis, gait analysis and physiological signals) or combinations thereof.

All of the data and information can be used to provide a score, e.g., in the scale of 1 to 10 how likely the participants are experiencing anxiety, depression and/or other human feeling and mental health issues. AI models and AI machine learning with training is used for this technology.

### 4.1 Data Collection

Platform 110 should be compliant with HIPPAA and GDPR and any other data regulations to protect and secure user's personal and health data. Platform 110 uses the millions of data points collected like users physical, emotional and behavioral patterns and users personal, professional and health data for scientific, research and development purposes to support socio-economic development of the country and community where blue group has its presence. The data points will also be used to improve people's quality of living, longevity and betterment of humanity. Platform 110 incorporates necessary tools and technologies to ensure their process is aligning to their vision and mission.

Platform 110 uses real-time device to device communications including and not limited to https and other transport and network protocols. On-demand therapy booking screens addresses the mental and emotional issues for users right when its needed using real-time communication platform. Data storage is secure cloud storage with geo-redundant storage and data replication. User system application 132 allows interlacing of live physical movement data from users phone/mobile devices (130) along with real time audio video streaming. Additionally, the technology platform 110 allows a strong scheduling algorithm to decide and allocate jobs (also known man as sessions) to relevant and available therapists. All of this happens in real time. The platform 110 also uses AI models to process the user entered text using natural language processing models and machine learning to find out patterns like mood, emotions, safety, security, maturity and modalities needed and ability to decide connect and involve government agencies like emergency services.

Platform 110 can support group chats. These can be live chat rooms with therapist being the group lead. This group chats allow the patients or clients of the same therapist to interact with each other for peer to peer support. Therapist and AI models are deployed for group therapy as needed. All group chats also support live real-time walking leaderboards for all the participants of the group. This enables “walking together” and peer to peer emotional support as part of the group therapy. The platform enables billing in a fashion similar to UBER ride sharing billing, that is, the users are billed right after the therapy session. Therapists are in control of how much to bill the user. Therapist can have 3 options for billing the user. Fully paid to denote the session was completed fully, partially paid to say the user ended the session or is incomplete for certain reasons or fully waived because the user did not attend the session. The tech platform enables automatic receipts and monthly subscription services for the group therapy option.

The above description of the disclosed embodiments is provided to enable any person skilled in the art to make or use the invention. Various modifications to these embodiments will be readily apparent to those skilled in the art, and the general principles described herein can be applied to other embodiments without departing from the spirit or scope of the invention. Thus, it is to be understood that the description and drawings presented herein represent a presently preferred embodiment of the invention and are therefore representative of the subject matter which is broadly contemplated by the present invention. It is further understood that the scope of the present invention fully encompasses other embodiments that may become obvious to those skilled in the art and that the scope of the present invention is accordingly not limited.

As used herein, the terms “comprising,” “comprise,” and “comprises” are open-ended. For instance, “A comprises B” means that A may include either: (i) only B; or (ii) B in combination with one or a plurality, and potentially any number, of other components. In contrast, the terms “consisting of,” “consist of,” and “consists of” are closed-ended. For instance, “A consists of B” means that A only includes B with no other component in the same context.

Combinations, described herein, such as “at least one of A, B, or C,” “one or more of A, B, or C,” “at least one of A, B, and C,” “one or more of A, B, and C,” and “A, B, C, or any combination thereof” include any combination of A, B, and/or C, and may include multiples of A, multiples of B, or multiples of C. Specifically, combinations such as “at least one of A, B, or C,” “one or more of A, B, or C,” “at least one of A, B, and C,” “one or more of A, B, and C,” and “A, B, C, or any combination thereof” may be A only, B only, C only, A and B, A and C, B and C, or A and B and C, and any such combination may contain one or more members of its constituents A, B, and/or C. For example, a combination of A and B may comprise one A and multiple B's, multiple A's and one B, or multiple A's and multiple B's.

What is claimed is:

1. A method comprising using at least one hardware processor to:
receive an indication for desire for a session with a professional;
confirm a professional's availability;
schedule session;
receive indication to begin session through a platform;
activate virtual walking video meeting, by activating video call;
receive information at least one of a pedometer and activity tracker; and
communicate information to the platform.

2. The method of claim 1, further comprising starting a timer when the virtual walking video meeting is activated.

3. The system of claim 1, further comprising receiving GPS information and communicating the GPS information to the platform.

4. The system of claim 1 further comprising receiving at least one of a user's live steps, distance, active hours, floors, calories and other vitals like weight, height, blood pressure, heart rate, pulse, walking pace, and wheel chair motions information and communicate such information to the platform.

5. A method comprising using at least one hardware processor to:
receive an indication for desire for a session with a user;
confirm a professional's availability;
schedule session;
receive indication to begin session through a platform;
activate virtual walking video meeting, by activating video call;
receive information at least one of a pedometer and activity tracker from a user device through the platform.

6. The method of claim 5, further comprising starting a timer when the virtual walking video meeting is activated.

7. The system of claim 1, further comprising receiving user GPS information through the platform.

8. The system of claim 1 further comprising receiving at least one of a user's live steps, distance, active hours, floors, calories and other vitals like weight, height, blood pressure, heart rate, pulse, walking pace, and wheel chair motions information through the platform.
